(19) United States
(12) Patent Application Publication
(10) Pub. No.: US 2024/0085723 A1
(43) Pub. Date: Mar. 14, 2024

(54) A VISION CORRECTIONAL LENS SUPPORTING ATTACHMENT MECHANISM

(71) Applicant: Masoud JAFARZADEH, Kew (AU)

(72) Inventor: Masoud JAFARZADEH, Kew (AU)

(21) Appl. No.: 18/261,777

(22) PCT Filed: Dec. 1, 2021
(86) PCT No.: PCT/AU2021/051433

§ 371 Date: Jul. 17, 2023

(30) Foreign Application Priority Data
Jan. 21, 2021 (AU) 2021900131

(51) Int. Cl. G02C 3/02; G02C 7/06

(52) U.S. Cl. CPC G02C 3/02; G02C 7/06; G02C 2200/02

(57) ABSTRACT

A vision correctional lens attachment has an attachment mechanism supporting a primary corrective lens vertically thereunderneath. The attachment mechanism is attachable to hold the primary lens a distance away from the eyes of the user for correction vision ahead therethrough. The present attachment has various applications, including for attachment to vehicle visors, helmets, goggles (such as ski goggles, scuba masks) and the like for correction of vision without having to wear glasses or contact lenses.

### FIELD OF THE INVENTION

This invention relates generally to a vision correctional lens attachment.

### SUMMARY OF THE DISCLOSURE

There is provided herein a vision correctional lens attachment comprising an attachment mechanism supporting a primary corrective lens vertically thereunderneath. The attachment mechanism is attachable to hold the primary lens a distance away from the eyes of the user for correction vision ahead therethrough.

The present attachment has various applications, including for attachment to vehicle visors, helmets, goggles (such as ski goggles, scuba masks) and the like for correction of vision without having to wear glasses or contact lenses.

In embodiments, the attachment mechanism comprises a clamp for attachment to a proximal and distal edges of the vehicle visors.

The corrective lens may have curvature having a focal point substantially at the eyes of the user in use. Furthermore, the attachment mechanism may comprise an offset adjustment mechanism to set the distance of the corrective lens from the eyes of the user to control the focal point.

The corrective lens may be a prescription lens having corrective geometry (such as one having a spherical, cylindrical or axial component), either integrally formed or applied by way of an adhesive overlay.

The primary corrective lens may be multifocal and, for example, comprise an upper far vision focal region and a lower near vision focal region. For vehicle use, the boundary therebetween may coincide substantially at the apparent junction between the windshield and dashboard. In embodiments, the height of the attachment may be adjusted or the attachment itself may allow for vertical offset adjustment so that the boundary between the focal regions may be set to coincide at the apparent junction between the windshield and the dashboard.

For use in vehicles, the attachment may comprise peripheral corrective lenses which may be hingedly coupled to sides of the primary corrective lens. The angle of the peripheral corrective lenses may be adjusted with respect to the primary corrective lens and hinges therebetween may allow the peripheral corrective lenses to move when forced but otherwise hold the peripheral corrective lenses in position with respect to the primary corrective lens.

The peripheral corrective lenses may similarly be prescription lenses, either comprising integrally formed correctional geometry or having an adhesive overlay having correctional geometry.

The peripheral corrective lenses may have different focal lenses as compared to the primary corrective lens and, in embodiments, as compared to each other. As such, for example, the primary corrective lens may comprise a far vision focal length for view of the road ahead, a passenger-side peripheral corrective lens may comprise a near vision focal length focusing on adjacent passengers and a driver's side peripheral corrective lens may comprise an intermediate focal length focusing on exterior objects adjacent the vehicle and the rear-view mirror.

The peripheral corrective lenses may themselves be multifocal but wherein, whereas the primary corrective lens may comprise horizontally orientated focal regions, the peripheral corrective lenses may comprise horizontally adjacent focal regions. For example, a driver-side peripheral corrective lens may comprise a rearward intermediate length focal length for focusing on exterior objects through the window and a forward near vision focal length for focusing on the rear-view mirror.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 illustrates installation of a vision correctional lens attachment in a vehicle in accordance with an embodiment;

FIG. 2 shows the vision correctional lens attachment of FIG. 1;

FIGS. 3 and 4 show installation of the vision correctional lens attachment in a cycle helmet according to an embodiment; and

FIGS. 5 and 6 show installation of the vision correctional lens attachment in the motorcycle helmet according to the further embodiment.

### DESCRIPTION OF EMBODIMENTS

A correctional lens attachment comprises an attachment mechanism 101 supporting a primary corrective lens 102 vertically thereunderneath. The attachment mechanism 101 is attachable to hold the primary lens a distance away from the eyes of the user for correction of vision ahead therethrough in use.

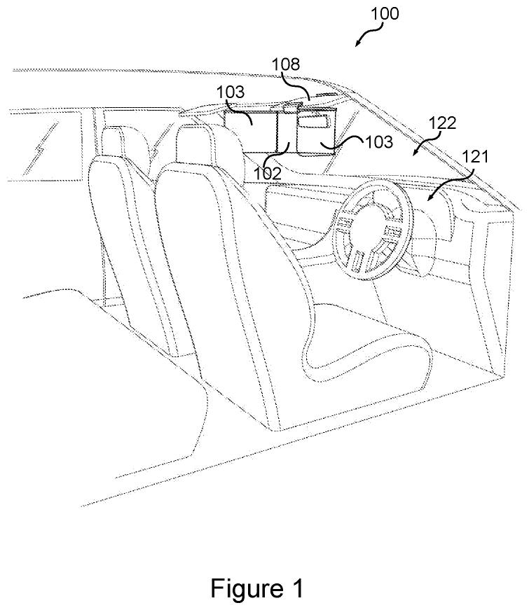

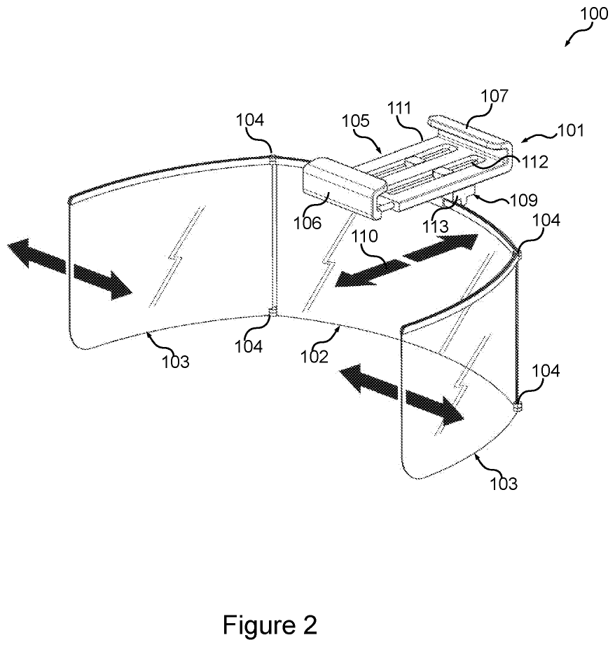

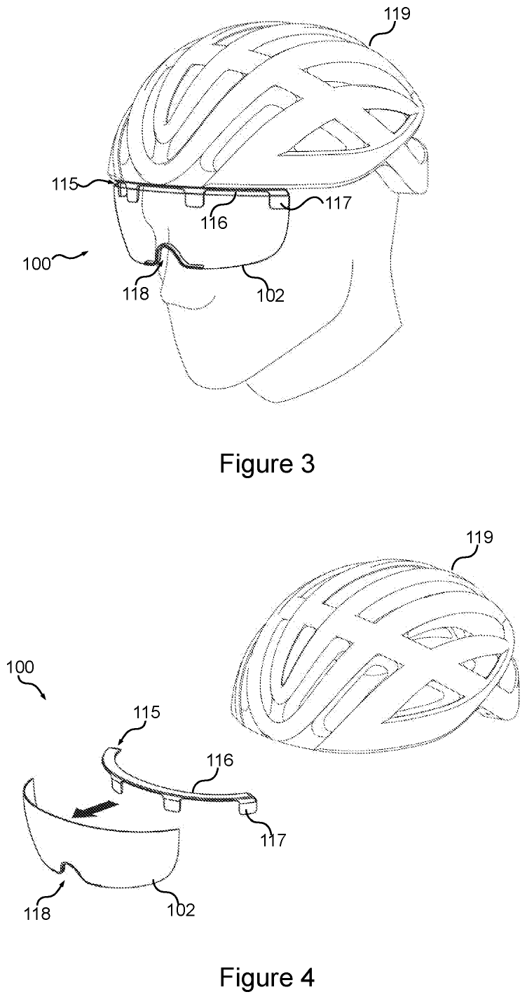

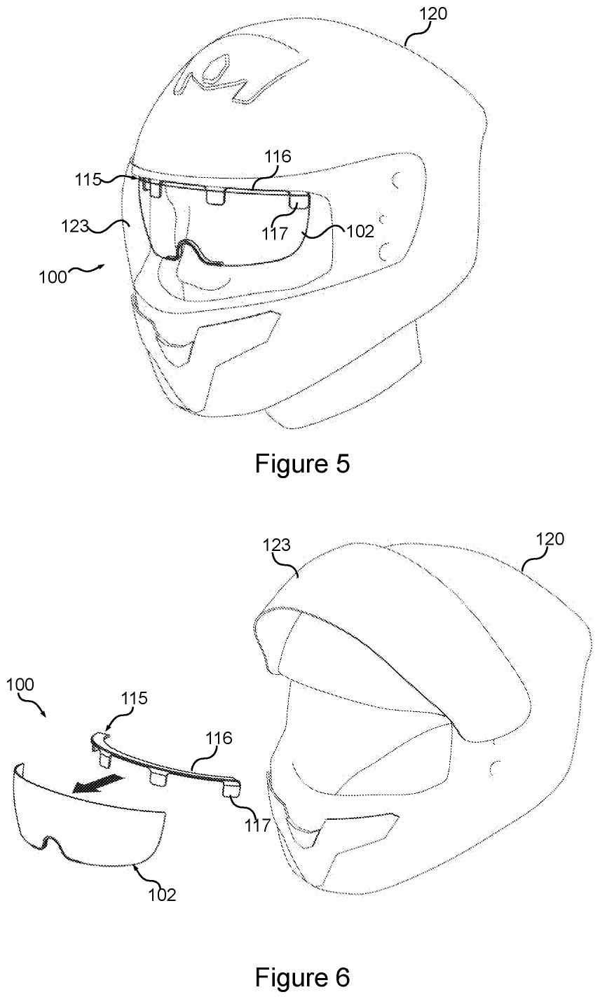

In the first embodiment shown in FIGS. 1 and 2, the correctional lens attachment 100 is for a vehicle, in the second embodiment shown in FIGS. 3 and 4, the correctional lens attachment 100 is suitable for cyclists and in the third embodiment shown in FIGS. 5 and 6, the correctional lens attachment 100 is suitable for motorcyclists and the like. The correctional lens attachment 100 may have other applications for correction of vision, including for scuba masks, ski goggle and the like.

With reference to the first embodiment, the primary corrective lens 102 may be generally rectangular. Furthermore, the primary corrective lens 102 may have curvature in horizontal cross section and wherein edges thereof curve towards the user. In this way, the curvature of the primary corrective lens 102 they have a focal point substantially at the eyes of the user in use.

The primary corrective lens 102 may be a prescription lens. In an embodiment, the primary corrective lens 102 may be a neutral lens (i.e. comprising uniform geometry/thickness) and wherein an adhesive transparent plastic overlay (not shown) itself comprising corrective geometry according to a user prescription is adhered to the neutral lens. The corrective geometry may comprise at least one of a spherical, cylindrical and axial components according to the requirements of the user.

In further embodiments, the primary corrective lens 102 itself has the corrective geometry specific for the user.

In embodiments, the primary corrective lens 102 may be a multifocal lens. Specifically, the primary corrective lens 102 may comprise an upper far vision focus lens and a lower near vision focus lens.

With reference to the installation of the attachment 100 within a vehicle shown in FIG. 1, the upper far vision focus lens is suitable for far vision for the road ahead whilst the lower near vision focus lens suitable for new vision of the vehicle dashboard 121. The user may adjust the height of the attachment 100 so that the horizontal boundary between the upper and lower focal lenses coincide substantially with the visual boundary between the windshield 122 and the dashboard 121 of the vehicle. In embodiment, the attachment mechanism 101 may comprise a height adjustment mechanism (not shown) allowing the user to adjust the vertical position of the primary corrective lens 102 thereunderneath.

As shown in FIG. 2, the attachment 100 may further comprise a peripheral corrective lens 103 at a side of the primary corrective lens 102 for correction of peripheral vision therethrough. In the embodiment shown, the attachment 100 comprises a pair of peripheral corrective lenses 103 either side of the primary corrective lens 102 for driver-side and passenger-side peripheral vision correction.

The peripheral correctional lens 103 may be hingedly coupled to the primary corrective lens 102. Hinges 104 between the primary corrective lens 102 and the peripheral corrective lenses 103 may allow the peripheral corrective lens 103 to move with respect to the primary corrective lens 102 when force is applied thereto but which otherwise hold the peripheral corrective lens 103 in position with respect to the primary corrective lens 102.

The peripheral corrective lens 103 may itself comprise curvature in a horizontal plane wherein side edges thereof curve towards the user so that such that the peripheral corrective lens 103 has a focal point substantially at the eyes of the user in use. Furthermore, the height of the peripheral corrective lens 103 may be substantially the same as that of the primary corrective lens 102.

The peripheral corrective lenses 103 may comprise different focal lengths as that of the primary corrective lens 102. In embodiments, each peripheral corrective lens 103 may comprise a different focal length. For example, the primary corrective lens 102 may comprise a focal length for far vision for the road ahead, the passenger side peripheral corrective lens 103 may comprise a near vision focal length for focusing on the passenger and passenger seat whereas the driver-side peripheral corrective lens 103 may comprise intermediate focal length of focusing on objects adjacent the vehicle, the rear-view mirror and the like.

Similarly, the peripheral corrective lenses 103 may be prescription lenses which may comprise integral correction geometry or alternatively comprise the aforedescribed adhesive layer applied thereto having the correctional geometry.

The peripheral corrective lenses 103 may themselves be multifocal in comprising comprise horizontally adjacent focal regions. For example, a driver-side peripheral corrective lens 103 may comprise a rearward intermediate length focal length for focusing on exterior objects through the window and a forward near vision focal length focusing on the rear-view mirror.

As is shown in FIG. 2, the attachment mechanism 101 may comprise a clamp 105 having a proximal claw 106 and a distal claw 107 movable apart and biased together the clamp respective proximal and distal edges of a vehicle visor 108 in the manner illustrated in FIG. 1. Furthermore, the attachment mechanism 101 may comprise an angle adjustment mechanism 109 interfacing the clamp 105 and the primary corrective lens 102 to adjust the angle of the primary corrective lens 102 with respect to the visor 108. Specifically, as is shown in FIG. 1, whereas the visor 108 may be at an angle, the angle adjustment mechanism 109 may be adjusted so that the primary corrective lens 102 thereunderneath is substantially vertical.

As is further shown in FIG. 2, the attachment mechanism 101 may further comprise an offset adjustment mechanism for adjusting the offset 110 of the primary corrective lens 102 from the eyes of the user in use. In this regard, the attachment mechanism 101 may comprise a static piece 111 comprising one or more longitudinal channels 112 arrange generally perpendicular with respect to a plane of the primary corrective lens 102 and a respective movable piece 113 comprising one or more respective engagements 114 engaged within the respective channels 112 and positionable at various positions within the channels 112.

With reference to the second and third embodiments shown in FIGS. 3 and 4, the attachment may comprise a forehead attachment rail 115 comprising a curved member 116 comprising a plurality of orthogonally orientated attachment tabs 117 extending thereunderneath.

The forward attachment rail 115 may be attached to a front underside edge of a cyclist helmet 119 in the manner shown in FIG. 3. Conversely, in the manner shown in FIG. 5, the forehead attachment rail 115 may be attached to a front underside edge of a motorcycle helmet 120.

The primary corrective lens 102 attaches to the attachment tabs 117. In embodiment, the attachment tabs 117 and primary corrective lens 102 attach magnetically. In accordance with this embodiment, the tabs 117 may comprise permanent magnets therein whereas the primary corrective lens 102 comprises a collocating metal piece which is magnetically attracted by the permanent magnets. As is also shown in FIG. 4, the primary corrective lens may comprise an ergonomic curvature and shape, including a nose bridge allowance 118.

In an embodiment, the correctional lens 102 may be provided as an adhesive overlay which is adhered directly to a vehicle windshield 122 or helmet face shield.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

The term “approximately” or similar as used herein should be construed as being within 10% of the value stated unless otherwise indicated.

1. A vision correctional lens attachment comprising an attachment mechanism supporting a primary corrective lens vertically thereunderneath, the attachment mechanism attachable to hold the primary lens a distance away from eyes of the user for correction of vision ahead therethrough.

2. The attachment as claimed in claim 1, wherein the primary lens is generally rectangular.

3. The attachment as claimed in claim 1, wherein the primary lens has curvature in horizontal cross section and wherein side edges thereof curve towards the user.

4. The attachment as claimed in claim 3, wherein the curvature has a focal point substantially at the eyes in use.

5. The attachment as claimed in claim 1, wherein an adhesive overlay comprising corrective geometry according to a user prescription is adhered to the primary lens.

6. The attachment as claimed in claim 5, wherein the corrective geometry comprises at least one of spherical component, a cylindrical component and an axial component.

7. The attachment as claimed in claim 1, wherein the primary corrective lens is a prescription lens and wherein the primary corrective lens itself comprises corrective geometry accordingly.

8. The attachment as claimed in claim 7, wherein the corrective geometry comprises at least one of spherical component, a cylindrical component and an axial component.

9. The attachment as claimed in claim 1, wherein the primary lens is a multi-focal lens.

10. The attachment as claimed in claim 9, wherein the primary lens comprises an upper far vision focus lens and a lower near vision focus lens.

11. The attachment as claimed in claim 1, further comprising a peripheral corrective lens at a side of the primary corrective lens for correction of peripheral vision therethrough.

12. The attachment as claimed in claim 11, wherein the peripheral corrective lens is hingedly coupled to the side.

13. The attachment as claimed in claim 12, wherein a hinge between the primary corrective lens and the peripheral corrective lens allows movement when force is applied to the peripheral corrective lens which otherwise holds the peripheral corrective lens in position with respect to the primary corrective lens.

14. The attachment as claimed in claim 11, wherein the peripheral corrective lens has curvature in a horizontal plane and wherein side edges thereof curve towards the user.

15. The attachment as claimed in claim 14, wherein the curvature has a focal point substantially at the eyes in use.

16. The attachment as claimed in claim 11, wherein the peripheral corrective lens has a height substantially the same as that of the primary corrective lens.

17. The attachment as claimed in claim 11, further comprising a further peripheral corrective lens at an opposite side of the primary corrective lens.

18. The attachment as claimed in claim 11, wherein the peripheral corrective lens has a different focal length from that of the primary corrective lens.

19. The attachment as claimed in claim 17, wherein the further peripheral corrective lens has a different focal length from that of the peripheral corrective lens.

20. The attachment as claimed in claim 17, wherein the primary corrective lens has a far vision focal length, the peripheral corrective lens has an intermediate distance focal length and the further peripheral corrective lens has a near vision focal length.

21. The attachment as claimed in claim 11, wherein the peripheral corrective lens comprises horizontally adjacent focal regions.

22. The attachment as claimed in claim 21, wherein the peripheral corrective lens comprises a rearward intermediate length focal length and a forward near vision focal length.

23. The attachment as claimed in claim 1, wherein the attachment mechanism comprises a clamp having proximal and distal claws movable apart and biased together to clamp respective proximal and distal edges of a vehicle visor.

24. The attachment as claimed in claim 23, wherein the attachment mechanism comprises an angle adjustment mechanism interfacing the clamp and the primary corrective lens.

25. The attachment as claimed in claim 1, wherein the attachment mechanism comprises an offset adjustment mechanism for adjusting the offset of the primary corrective lens from the eyes in use.

26. The attachment as claimed in claim 25, wherein the attachment mechanism comprises a static piece and a movable piece slidably engaged to the static piece.

27. The attachment as claimed in claim 26, wherein the static piece comprises longitudinal channels arranged generally perpendicular with respect to a plane of the primary lens and wherein the movable piece comprises engagements slidably engaged therein and positionable at various positions within the channels.

28. The attachment as claimed in claim 27, wherein the static piece comprises a clamp having proximal and distal edges movable apart and biased together to clamp respective proximal and distal edges of a visor.

29. The attachment as claimed in claim 1, wherein the attachment comprises a forehead attachment rail comprising a curved member comprising a plurality of orthogonally orientated attachment tabs extending thereunderneath and wherein the primary corrective lens attaches to the attachment tabs.

30. The attachment as claimed in claim 29, wherein the attachment tabs and primary corrective lens attach magnetically.

31. The attachment as claimed in claim 30, wherein the tabs comprise permanent magnets therein whereas the primary corrective lens comprises a collocating metal piece which is magnetically attracted by the permanent magnets.
